(19) United States
(12) Patent Application Publication
(10) Pub. No.: US 2020/0229734 A1
(43) Pub. Date: Jul. 23, 2020

(54) METHOD AND ARRANGEMENT FOR DETERMINING A NON-THERAPEUTIC TREATMENT OF THE ORAL CAVITY

(71) Applicant: Henkel AG & Co. KGaA, Duesseldorf (DE)

(72) Inventors: ANDREAS BOCK, Nuess (DE); THOMAS WELSS, Duesseldorf (DE); KRISTIN MIEHLICH, Wuppertal (DE); CLAUDIA HUNDEIKER, Meerbusch (DE); MATTHIAS FRISCHMANN, Duesseldorf (DE)

(73) Assignee: Henkel AG & Co. KGaA, Duesseldorf (DE)

(21) Appl. No.: 16/489,741

(22) PCT Filed: Feb. 15, 2018
(86) PCT No.: PCT/EP2018/053778

§ 371 Date: Aug. 29, 2019

(30) Foreign Application Priority Data
Mar. 3, 2017 (DE) 10 2017 203 499.8

(51) Int. Cl. A61B 5/08; G01N 33/497

(52) U.S. Cl. CPC A61B 5/082; G01N 2033/4975; G01N 33/497

(57) ABSTRACT

The present disclosure relates to various exemplary embodiments of a method for determining a non-therapeutic treatment of the oral cavity, wherein the method comprises measuring volatile organic constituents in air exhaled from the oral cavity, determining a respiratory-air load on the basis of the measured concentration, and determining a non-therapeutic treatment of the oral cavity on the basis of the determined respiratory-air load.

### CROSS-REFERENCE TO RELATED APPLICATION

This application is a U.S. National-Stage entry under 35 U.S.C. § 371 based on International Application No. PCT/EP2018/053778, filed Feb. 15, 2018, which was published under PCT Article 21(2) and which claims priority to German Application No. 10 2017 203 499.8, filed Mar. 3, 2017, which are all hereby incorporated in their entirety by reference.

### TECHNICAL FIELD

The present disclosure relates to methods and assemblies for determining a non-therapeutic treatment of the oral cavity.

### BACKGROUND

In many areas of daily life there has been a trend for some time now for personalised programmes which specifically address individual requirements and needs (for example in the fields of nutrition or health). This is also true, however, for personalised cosmetics. Here, the objective is to make it possible for the consumer to find products in a very targeted manner or to obtain care-related suggestions which are tailored to the individual needs of the consumer. Accordingly, user-friendly and effective approaches for determining a non-therapeutic treatment, for example of the oral cavity, are desirable.

### BRIEF SUMMARY

In accordance with various exemplary embodiments a method for determining a non-therapeutic treatment of the oral cavity is provided, wherein the method comprises measuring volatile organic constituents in air exhaled from the oral cavity, determining a respiratory-air load on the basis of the measured concentration, and determining a non-therapeutic treatment of the oral cavity on the basis of the determined respiratory-air load.

In accordance with a further exemplary embodiment an assembly for determining a non-therapeutic treatment of the oral cavity is provided.

In accordance with an embodiment, a method for monitoring the success of a non-therapeutic treatment of the oral cavity is additionally provided.

### BRIEF DESCRIPTION OF THE DRAWINGS

The present disclosure will hereinafter be described in conjunction with the following drawing figures, wherein like numerals denote like elements, and wherein:

FIG. 1 shows an assembly for determining a non-therapeutic treatment of the oral cavity.

FIG. 2 shows a graph showing the respiratory-air load of a user over time.

FIG. 3 shows a flow diagram illustrating a method for determining a non-therapeutic treatment of the oral cavity.

FIG. 4 shows an assembly for determining a non-therapeutic treatment of the oral cavity.

FIG. 5 shows a method for monitoring the success of a non-therapeutic treatment of the oral cavity.

### DETAILED DESCRIPTION

In the following detailed description, reference is made to the accompanying drawings, which form part of the present application and in which specific embodiments in which the present disclosure can be carried out are shown by way of illustration. It goes without saying that other embodiments can be used and structural or logical changes can be made without departing from the scope of protection of the present disclosure. It goes without saying that the features of the various exemplary embodiments described herein can be combined with one another unless specifically stated otherwise. The following detailed description is therefore not to be interpreted in a limiting sense, and the scope of protection of the present disclosure is defined by the accompanying claims.

Bad breath (halitosis) us produced by the breakdown of food constituents by bacteria. These are usually organic and sulphurous, highly volatile substances, which lead to malodorous breath. Approximately 1000 different volatile compounds that, in the case of bad breath in people, can be mixed with the exhaled respiratory air have been identified by employing gas chromatography. These include, inter alia, sulphur compounds, such as hydrogen sulphide (H₂S), methanethiol (methyl mercaptan) and dimethyl sulphide as well as amines, diamines and other nitrogen compounds, such as 1,5-diaminopentane, indole and skatole, ketones, such as acetone (propanone), and short-chain carboxylic acids (propionic acid, butyric acid). These substances are formed for example by the bacterial breakdown of organic substances from food residues, saliva or dead tissue material (desquamated epithelial cells).

People who suffer from bad breath usually cannot themselves detect the severity of the odour. Those affected, however, are often excluded and are placed under social pressure.

Bad breath can be detected, however, by use of a sensor that specifically measures volatile organic constituents (VOCs) in the breath, for example one or more of the above-mentioned volatile compounds. In accordance with various embodiments a respiratory-air load of a user is determined in this way, and a non-therapeutic treatment can be determined on the basis of the determined respiratory-air load and can be proposed to the user.

Volatile organic compounds (VOCs) is the collective term for organic, that is to say carbonaceous, substances which evaporate easily (are volatile) or are present in the form of a gas already at low temperatures (for example room temperature).

The respiratory-air load will be understood in the following to be the odour loading of the exhaled air. In other word, respiratory-air load means that substances (such as those mentioned above) which are considered to be malodorous are present in the exhaled air from a person, moreover in a significant amount, such that they become apparent in the form of (noticeable) bad breath.

For example a Sniffer study can be used to determine whether a sample falls into the category of noticeable bad breath. Sniffers can be trained assessors, who for example undergo regular training by an institute and are then selected according to their suitability. The odour threshold of the tester can be checked by employing dynamic olfactometry. Accordingly, concentrations of VOCs in the exhaled air can be assigned bad breath ratings. Assuming, for example, 1-1000 ppb (parts per billion) VOCs in exhaled air, value ranges of VOC concentrations can be assigned respiratory-air load grades (i.e. bad breath grades) in accordance with an assessment by the Sniffers, shows for example in Table 1.

TABLE 1

| Concentration VOC in ppb | Bad breath rating |
|---|---|
| 1-500 | No bad breath |
| 501-700 | Slight bad breath |
| 701-900 | Medium bad breath |
| 901-1000 | Strong bad breath |

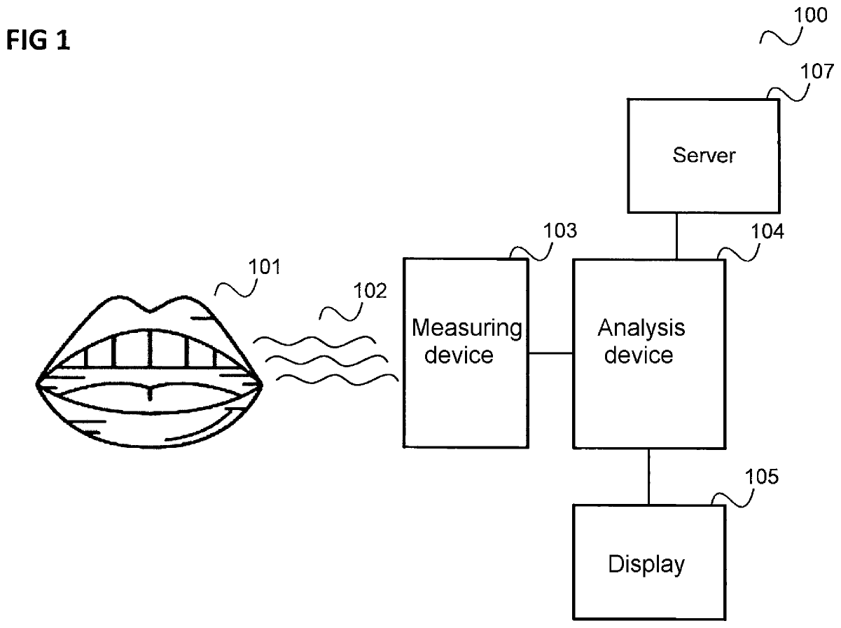

FIG. 1 shows an assembly for determining a non-therapeutic treatment of the oral cavity.

The concentration of VOCs in the air 102 exhaled from the mouth 101 of a user (breath) is measured by a measuring device 103.

The measuring device 103 uses a technique for quantifying VOCs in the respiratory air. For example the device comprises a selective sensor which specifically measures one or more volatile organic constituents or also groups of volatile organic constituents (VOCs) in the breath 102.

The measuring device 103 can measure the VOC concentration for example with use of a redox sensor, a photoionisation detection (PID) sensor or on the basis of gas chromatography (GC) or a combination thereof.

An analysis device 104 is coupled to the measuring device 103. The analysis device 104 (and optionally also the measuring device 103) can be, for example, an electronic apparatus, for example a portable electronic apparatus such as a smart phone, a smart belt, a smart watch, a PDA (personal digital assistant), a tablet computer (or integrated therein or realised thereby) or also any computer. The measuring device 103 can be of any size. In order to allow the user to carry the measuring device 103 on his person (and for example to use it with his smart phone), it can be of such a size for example that it fits without difficulty in handbags and trouser packets (for example footprint area<16 cm², height<1 cm).

The measuring device 103 is an apparatus for quantifying VOCs in the exhaled air 102 and for example measures the VOC concentration in the exhaled air 102 in a specific unit (for example ppb).

The analysis device 104, on the basis of the measured VOC concentration, determines a respiratory-air load, for example in the form of a respiratory air load value. For example, the analysis device 104 generates a value of the respiratory-air load in a random unit, for example a severity of the bad breath, or also a verbal instruction for the user on the basis of the measured VOC concentration. For example the analysis device 104 can determine a bad breath category as shown in Table 1 on the basis of the measured VOC concentration, possibly with more gradations than stated in Table 1.

The analysis device 104 can then determine a non-therapeutic treatment proposal on the basis of the determined respiratory-air load and for example can display this on a display 105 (for example the display of the smart phone or the tablet computer as appropriate) coupled to the analysis device 104. For example the assembly 100 comprises a screen, a display or a touchscreen in order to provide the user with information, for example with determined recommendations for a non-therapeutic treatment. The assembly 100 can also have an audio output for this purpose.

The analysis device for example can run an analysis software (for example an app) which performs the functions described above and also hereinafter.

For example, the analysis device 104 can deduce and display product recommendations for individual oral hygiene, for example recommendations for improving the individual dental care, for example teeth brushing.

If the determined respiratory-air load lies for example in a critical range, the analysis device 104 can for example suggest to the user how often he should brush his teeth or use mouthwash (possibly with suggestions as to which mouthwash the user could use or which toothpastes the user could use) or might even recommend a trip to the dentist. The recommendation of a trip to the dentist, for example for professional dental cleaning, can link the analysis device 104 directly to a software (for example an app), which makes it possible to schedule an appointment at a dentist. Further non-therapeutic treatments of the oral cavity can be the use of special toothbrushes, for example interdental brushes, toothbrushes for implants or dentures, tongue cleaners, agents, in particular gels, for application to the teeth and/or the gums, mouth rinses, mouth sprays and/or chewing gums.

The analysis device 104 can also output a warning to the user that the bad breath could lead to a negative reaction in the presence of other people if the determined respiratory-air load reaches a certain level (for example “medium bad breath” or “strong bad breath” according to the categorisation in Table 1).

The analysis device 104 can also give diet tips for improved breath as a treatment proposal.

If the respiratory-air load is uncritical (for example in the category “no bad breath”) the analysis device 104 can also display that everything is in order in this regard. The treatment proposal in this case can be considered to be that of maintaining current habits.

The treatment proposal can include a product recommendation that is dependent on the respiratory-air load (i.e. the level of the respiratory-air load).

In accordance with various exemplary embodiments the non-therapeutic treatment may additionally be determined on the basis of predefined data. the predefined data for example can be stored in a memory and/or can be provided from an external data processing facility. The predefined data can be present in the form of a database. The predefined data can contain literature data, for example relating to the compatibility and ingredients of cosmetics, or also data relating to courses of treatment followed by other consumers. Accordingly, the data can comprise information relating to cosmetics, for example their ingredients and their effects on body parts. The predefined data can be acquired upon the request of the user or regularly by an external data processing facility.

The analysis device 104 for example can have a communication interface for data transfer to a server 107 (or generally an external data processing facility). The server 107 can be contactable for example via the Internet and for example can be part of a server network which implements a cloud.

The measuring device 103 and the analysis device 104 are likewise connected by employing a communication interface.

The communication interfaces can be designed for example for wired or wireless communication. For example, data can be transferred by employing USB (universal serial bus), WLAN (wireless local area network), NFC (near-field communication), Thread, ZigBee and/or Bluetooth. The two communication interfaces can have corresponding communication circuits and/or can be coupled thereto.

The analysis device 104 can comprise a processor which is coupled to the communication interface(s) so that a data transfer can occur between the processor and the measuring device 103 or the server 107. The communication interfaces can also be integrated wholly or partially in the processor. The processor for example can be a microprocessor, a microcontroller or an ASIC (application-specific integrated circuit). The processor can be configured for example by employing software for the exemplary embodiments described above and hereinafter (also combined). For example, the software can be a mobile application, a web application or a desktop application. The software can be stored in a memory of the analysis device and/or at least in part in an external data processing facility, such as the server 107, and can be accessible to the processor. The analysis device, for example the processor thereof, for example controls and monitors the detection system for carrying out the measurement.

In order to assess the values measured by the measuring device 103, the analysis device 104 can compare the measurement information with information stored in a memory, for example a database, or with information which it acquires from the server 107. For example, the measurement information can be assigned to one or more categories, for example on the basis of predefined value ranges.

The analysis device 104 can optionally comprise an input unit. Data can be input and queried via the input unit and can be included in the determination of a non-therapeutic treatment. For example a user can specify an allergy and/or allergens known to him, and thus the recommendation of an allergen in the determination of a non-therapeutic treatment can be prevented. The analysis device 104 can request that the user input data by employing a corresponding display.

The determination of the respiratory-air load can be performed for a user over a longer period of time. The analysis device 104 can then record a status of the user's breath on the basis of the measured respiratory-air load over a longer period of time.

The analysis device 104 can then for example assess the success of a non-therapeutic treatment performed by the user in the period of time (and proposed to him by the analysis device 104 for example at the start of the period of time) in a standardised manner and objectively, for example at home, when the user for example uses his smart phone or his tablet computer for this purpose. This allows the user to monitor the individual efficacy of an oral hygiene product and/or oral hygiene and can (depending on success) increase his motivation to perform a corresponding treatment, even in the longer term.

For example, the analysis device 104 enables the monitoring and tracking of the respiratory-air load of a user by displaying the measurement results over the course of time.

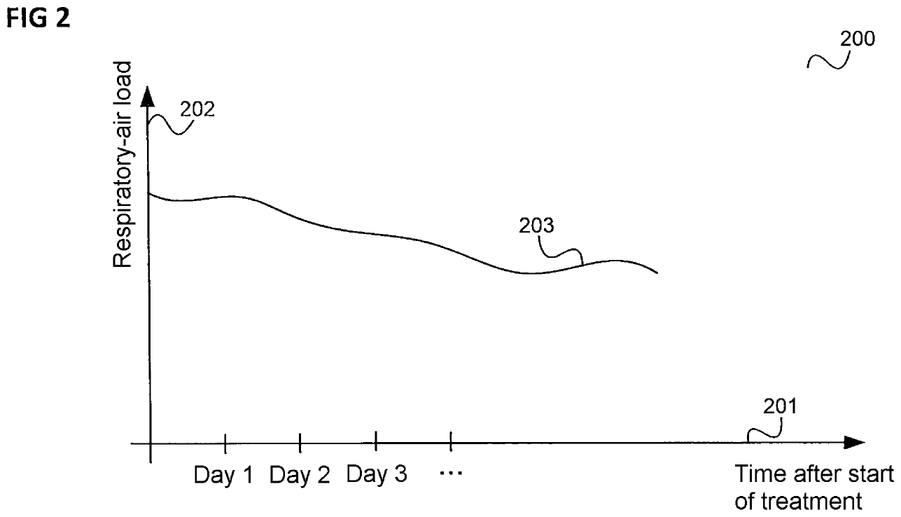

For example the analysis device 104 shows the course of the determined respiratory-air load in graph form, as shown in FIG. 2.

FIG. 2 shows a graph 200 illustrating the respiratory-air load of a user over time.

For example the analysis device 204 determines the respiratory-air load of a user each day over a period of time lasting several weeks. The days are plotted along the x-axis 201 and the respiratory-air load (in a certain unit, for example a VOC concentration) is specified in accordance with the scale of the y-axis 202. The values in this example are associated with a smoothed curve 203 in order to indicate continuous development. Depending on how often the user performs a measurement, the analysis device 104 can determine the respiratory-air load, even at shorter time intervals, and can generate the curve 203 on the basis of a higher number of (or denser) determined values of the respiratory-air load.

Depending on how the determined respiratory-air load develops over time, i.e. on account of the attained results of the non-therapeutic treatment, the analysis device 104 can again give care and product recommendations. If the treatment for example does not lead to any improvement, the analysis device 104 can recommend other products or products of a stronger dose.

The analysis device 104, when determining the non-therapeutic treatment (for example care and product recommendations), can also take into consideration answers provided by the user to questions regarding his general dental condition (for example presence of dentures, such as implants, bridges, etc., or braces), his oral hygiene habits (for example name of oral hygiene products used, frequency of teeth cleaning, use of special toothbrushes, such as interdental brushes or tongue brushes), his dietary habits, his general state of health (for example with regard to illnesses, allergies, medication intake) and further behaviour (for example smoking habits. For example the analysis device 104 can display corresponding questions on the display 105 and via an input device (for example a keypad or a touchscreen) can receive and appropriately process the answers, i.e. can determine the non-therapeutic treatment on the basis of the answers.

The analysis device 104, when determining the non-therapeutic treatment, can additionally base its decision on literature data and also the success of non-therapeutic treatments followed by other users (for example of the same software or app used by the analysis device 104).

For example the respiratory-air loads determined during the course of a non-therapeutic treatment are uploaded to a server 107 (for example a cloud), for example together with a profile of the user, and the analysis device 104 can download such data from other users, for example from users who have a similar profile to the user of the analysis device 104, in order to determine which treatment methods are successful for users of such a profile or a similar profile. It can also be determined server-side which treatment methods are successful for which user profiles, and the analysis device 104 can download this information and include it when determining the non-therapeutic treatment.

The user profile can include for example oral hygiene habits, eating habits, drinking habits, smoking habits, gender, height, age, weight, etc.

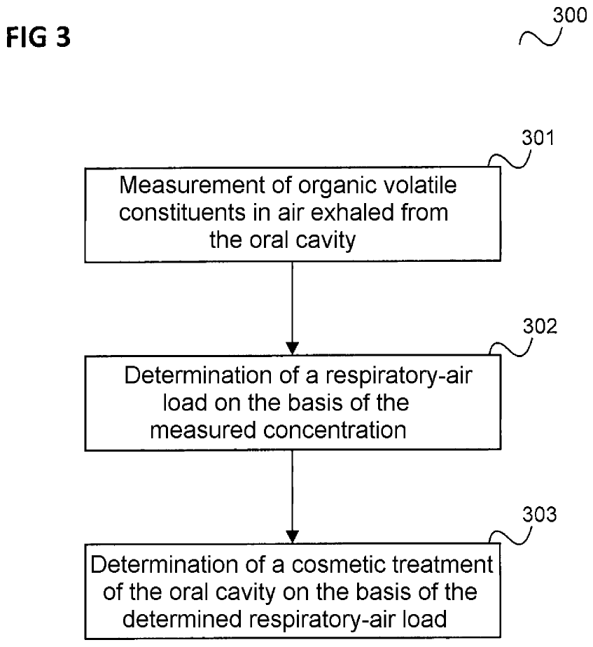

To summarise, a method as shown in FIG. 3 is provided in accordance with various embodiments.

FIG. 3 shows a flow diagram 300 which illustrates a method for determining a non-therapeutic treatment of the oral cavity.

In 301 volatile organic constituents in exhaled air from the oral cavity measured.

In 302 a respiratory-air load is determined on the basis of the measured concentration.

In 303 a non-therapeutic treatment of the oral cavity is determined on the basis of the determined respiratory-air load.

In other words, in accordance with various embodiments, the respiratory-air load of a user is measured by measuring the concentration of volatile organic constituents in the exhaled air, and a treatment proposal is determined on the basis of the respiratory-air load.

The method enables a user for example to obtain individual care recommendations and product recommendations which are tailored to his needs.

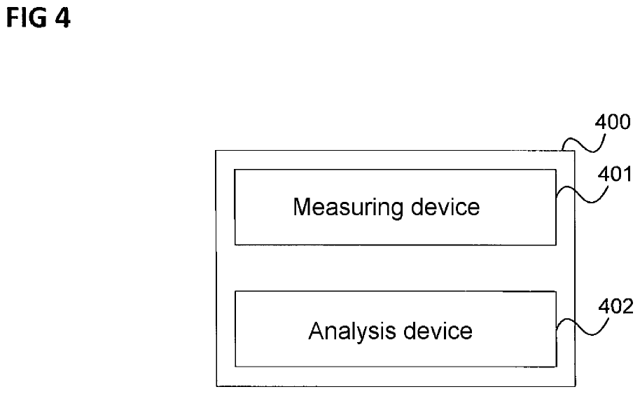

The method may be performed for example by an assembly as shown in FIG. 4.

FIG. 4 shows an assembly 400 for determining a non-therapeutic treatment of the oral cavity.

The assembly 400 comprises a measuring device 401, which is designed to measure volatile organic constituents in air exhaled from the oral cavity.

In addition, the assembly 400 comprises an analysis device 402, which is designed to determine a respiratory-air load on the basis of the measured concentration, and to determine a non-therapeutic treatment of the oral cavity on the basis of the determined respiratory-air load.

The measuring device 401 for example can be an attachment for a smart phone, and the analysis device 402 can be a smart phone. Alternatively, the measuring device 401 and the analysis device 402 can form an integrated unit, for example in that the measuring device 401 is fixedly installed with the analysis device 402. In various exemplary embodiments the measuring device 401 and the analysis device 402 separate units, which may communicate wirelessly, via communication interfaces.

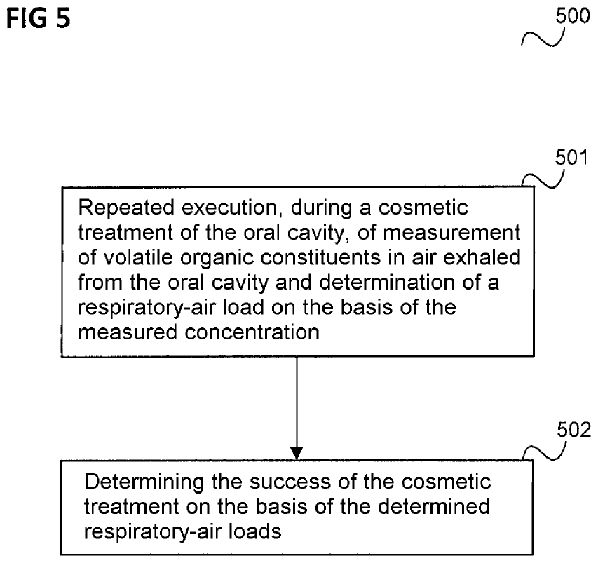

In accordance with a further embodiment a method is provided as shown in FIG. 5.

FIG. 5 shows a flow diagram 500 which illustrates a method for monitoring the success of a non-therapeutic treatment of the oral cavity.

In 501, during the non-therapeutic treatment of the oral cavity, volatile organic constituents in air exhaled from the oral cavity are measured and a respiratory-air load is determined on the basis of the measured concentration, these acts being performed repeatedly.

In 502 the success of the non-therapeutic treatment is determined on the basis of the determined respiratory-air load.

In other words, in accordance with an embodiment, the respiratory-air load according to FIG. 4 is determined continuously, i.e. for example repeatedly or at regular intervals, during a treatment period and the success of the treatment is assessed on the basis of the determined respiratory-air loads (for example respiratory-air load values).

The method according to FIG. 5 enables the monitoring and tracking of the efficacy of a non-therapeutic treatment objectively and in a standardised manner on the basis of the respiratory-air load. The efficacy of the treatment can be optimised by targeted product recommendations.

The user can be directly offered a recommended product for purchase, and the user can arrange to purchase the product by way of an input. Besides the purchase of recommended oral hygiene products, the user can also be offered more detailed information with regard to the purchase. This more detailed information can relate to more detailed treatment and use instructions. A software/app for example receives the request that the user would like to purchase the oral hygiene product, stores the request and/or transmits the request to a trade company that sells the oral hygiene products. The user can be requested by the software/app to input his personal data (address, bank information, shipping preferences, etc.) via the input unit. Alternatively, the user may be advised as to where he can acquire the oral hygiene product (for example drugstore, pharmacy, etc.). The software/app for example can be the same software or app used by the analysis device 104.

Increasing numbers of users are looking for a product tailored individually to their needs. This product may be a product produced especially for the user or what is known as a “mass customised” product. In the case of a “mass customised” product, an individualisation can be achieved by varying just a few features of a product, which features however are key from the customer perspective. These “mass customised” products are for example based on the concept of modularisation, that is to say the product can be compiled individually from various modules/building blocks.

There are often numerous dependencies between the many different features/ingredients of a product, and said dependencies can be expressed as “dos” and “don'ts”. In order to obtain a clear product definition, it can be advantageous that the ordering process is performed with the aid of a product configurator. This configurator helps the customer in his choice of features/ingredients and advises him of the admissible/inadmissible feature combinations, wherein the latter then cannot be selected.

In the case of oral hygiene products the relevant product features include in particular the chemical ingredients of the products, the physical properties of the products and the type of formulation of the products. With the aid of a product configurator, chemically and/or physically incompatible ingredients can be prevented from being selected, or ingredients that are unsuitable for the determined respiratory-air load can be prevented from being selected. Conversely, the selection of ingredients that are suitable for the determined respiratory-air load can be dictated or proposed by the product configurator.

Suitable ingredients of oral hygiene products such as tooth creams, mouth rinses, gels for application to the teeth and/or the gums, mouth sprays or chewing gums for combating bad breath comprise, for example, zinc acetate, chlorhexidine, fluorides, such as sodium fluoride, tin fluoride and/or amine fluorides, such as olaflur, essential oils, such as thymol, eucalyptol, menthol and/or methyl salicylate, zinc lactate, cetyl pyridinium chloride and/or zinc chloride. In addition, further ingredients that are essential and/or conventional for the oral hygiene product in question can also be contained.

The determination of a chemical composition of an oral hygiene product comprises in particular the determination of a chemical composition of an oral hygiene product in respect of the active substance(s) effective against bad breath. These active substances comprise, for example, zinc acetate, chlorhexidine, fluorides, such as sodium fluoride, tin fluoride and/or amine fluorides, such as olaflur, essential oils, such as thymol eucalyptol, menthol, and/or methyl salicylate, zinc lactate, cetyl pyridinium chloride and/or zinc chloride.

An overview of the success of a non-therapeutic treatment can increase the motivation of the user to perform the non-therapeutic treatment in the longer term—also by comparison with other users. For example, results from a number of users are loaded onto a server, so that each user can compare his own success of the treatment with the success of the treatment in other users.

Exemplary embodiments will be described hereinafter:

Exemplary embodiment 1 is a method for determining a non-therapeutic treatment of the oral cavity, as shown in FIG. 3.

Exemplary embodiment 2 is the method according to exemplary embodiment 1, wherein the respiratory-air load is an odour load.

Exemplary embodiment 3 is the method according to exemplary embodiment 1 or 2, wherein the measurement of volatile constituents includes the measurement of the concentration of volatile organic constituents in the exhaled air.

Exemplary embodiment 4 is the method according to any one of exemplary embodiments 1 to 3, wherein the measurement of volatile constituents includes the measurement of a concentration of one or more sulphur compounds and/or nitrogen compounds.

Exemplary embodiment 5 is the method according to any one of exemplary embodiments 1 to 4, wherein the measurement of volatile constituents includes the measurement of a concentration of volatile constituents by employing a redox sensor, a photoionisation detection (PID) sensor and/or on the basis of gas chromatography (GC) or a combination thereof.

Exemplary embodiment 6 is the method according to any one of exemplary embodiments 1 to 5, further comprising displaying a specification of the determined non-therapeutic treatment on a display.

Exemplary embodiment 7 is the method according to any one of exemplary embodiments 1 to 6, wherein the non-therapeutic treatment includes the use of an oral hygiene product.

Exemplary embodiment 8 is the method according to any one of exemplary embodiments 1 to 7, wherein the determination of the non-therapeutic treatment includes the determination of a chemical composition of an oral hygiene product.

Exemplary embodiment 9 is the method according to any one of exemplary embodiments 1 to 8, wherein the non-therapeutic treatment is determined on the basis of personal information relating to the user for whom the respiratory-air load is determined.

Exemplary embodiment 10 is the method according to exemplary embodiment 9, wherein the personal information includes one or more items of information regarding age, gender, height, weight, allergies/allergens and habits of the user.

Exemplary embodiment 11 is the method according to exemplary embodiment 9, wherein the personal information includes one or more items of information regarding eating habits, drinking habits, smoking habits, oral hygiene habits and used oral hygiene products.

Exemplary embodiment 12 is the method according to any one of exemplary embodiments 1 to 11, wherein the non-therapeutic treatment is determined on the basis of the success of one or more non-therapeutic treatments in one or more other users.

Exemplary embodiment 13 is the method according to exemplary embodiment 12, comprising selecting the one or more other users from a plurality of candidates on the basis of a comparison of personal information relating to the user with personal information relating to the candidates.

Exemplary embodiment 14 is the method according to any one of exemplary embodiments 1 to 13, including storing the determined respiratory-air load.

Exemplary embodiment 15 is the method according to any one of exemplary embodiments 1 to 14, including storing the determined respiratory-air load on a server.

Exemplary embodiment 16 is an assembly for determining a non-therapeutic treatment of the oral cavity as is shown in FIG. 4.

Exemplary embodiment 17 is the assembly according to exemplary embodiments 16, wherein the respiratory-air load is an odour load.

Exemplary embodiment 18 is the assembly according to exemplary embodiments 16 or 17, wherein the measurement of volatile constituents includes the measurement of a concentration of volatile organic constituents in the exhaled air.

Exemplary embodiment 19 is the assembly according to any one of exemplary embodiments 16 to 18, wherein the measurement of volatile constituents includes the measurement of a concentration of one or more sulphur compounds and/or nitrogen compounds.

Exemplary embodiment 20 is the assembly according to any one of exemplary embodiments 16 to 19, wherein the measurement of volatile constituents includes the measurement of a concentration of volatile constituents by employing a redox sensor, a photoionisation detection (PID) sensor and/or on the basis of gas chromatography (GC) or a combination thereof.

Exemplary embodiment 21 is the assembly according to any one of exemplary embodiments 16 to 20, further comprising displaying a specification of the determined non-therapeutic treatment on a display.

Exemplary embodiment 22 is the assembly according to any one of exemplary embodiments 16 to 21, wherein the non-therapeutic treatment includes the use of an oral hygiene product.

Exemplary embodiment 23 is the assembly according to any one of exemplary embodiments 16 to 22, wherein the determination of the non-therapeutic treatment includes the determination of a chemical composition of an oral hygiene product.

Exemplary embodiment 24 is the assembly according to any one of exemplary embodiments 16 to 23, wherein the non-therapeutic treatment is determined on the basis of personal information relating to the user for whom the respiratory-air load is determined.

Exemplary embodiment 25 is the assembly according to exemplary embodiment 24, wherein the personal information includes one or more items of information regarding age, gender, height, weight, allergies/allergens and habits of the user.

Exemplary embodiment 26 is the assembly according to exemplary embodiment 24, wherein the personal information includes one or more items of information regarding eating habits, drinking habits, smoking habits, oral hygiene habits and used oral hygiene products.

Exemplary embodiment 27 is the assembly according to any one of exemplary embodiments 16 to 26, wherein the non-therapeutic treatment is determined on the basis of the success of one or more non-therapeutic treatments in one or more other users.

Exemplary embodiment 28 is the assembly according to exemplary embodiment 27, comprising selecting the one or more other users from a plurality of candidates on the basis of a comparison of personal information relating to the user with personal information relating to the candidates.

Exemplary embodiment 29 is the assembly according to any one of exemplary embodiments 16 to 28, including storing the determined respiratory-air load.

Exemplary embodiment 30 is the assembly according to any one of exemplary embodiments 16 to 29, including storing the determined respiratory-air load on a server.

Exemplary embodiment 31 is a method for monitoring the success of a non-therapeutic treatment of the oral cavity as shown in FIG. 5.

Exemplary embodiment 32 as the method according to exemplary embodiment 31, further including displaying a specification of the determined success on a display.

Exemplary embodiment 33 is the method according to exemplary embodiment 31 or 32, wherein the respiratory-air load is an odour load.

Exemplary embodiment 34 is the method according to any one of exemplary embodiments 31 to 33, wherein the measurement of volatile constituents includes the measurement of a concentration of volatile organic constituents in the exhaled air.

Exemplary embodiment 35 is the method according to any one of exemplary embodiments 31 to 34, wherein the measurement of volatile constituents includes the measurement of a concentration of one or more sulphur compounds and/or nitrogen compounds.

Exemplary embodiment 36 is the method according to any one of exemplary embodiments 31 to 35, wherein the measurement of volatile constituents includes the measurement of a concentration of volatile constituents by employing a redox sensor, a photoionisation detection (PID) sensor and/or on the basis of gas chromatography (GC) or a combination thereof.

Exemplary embodiment 37 is the method according to any one of exemplary embodiments 32 to 36, further comprising displaying a specification of the non-therapeutic treatment on a display.

Exemplary embodiment 38 is the method according to any one of exemplary embodiments 31 to 37, wherein the non-therapeutic treatment includes the use of an oral hygiene product.

Exemplary embodiment 39 is the method according to any one of exemplary embodiments 31 to 38, wherein the non-therapeutic treatment includes the use of an oral hygiene product having a specific chemical composition.

Exemplary embodiment 40 is the method according to any one of exemplary embodiments 31 to 39, wherein the non-therapeutic treatment is determined on the basis of personal information relating to the user for whom the respiratory-air load is determined.

Exemplary embodiment 41 is the method according to exemplary embodiment 40, wherein the personal information includes one or more items of information regarding age, gender, height, weight, allergies/allergens and habits of the user.

Exemplary embodiment 42 is the method according to exemplary embodiment 40, wherein the personal information includes one or more items of information regarding eating habits, drinking habits, smoking habits, oral hygiene habits and used oral hygiene products.

Exemplary embodiment 43 is the method according to any one of exemplary embodiments 31 to 42, wherein the non-therapeutic treatment is determined on the basis of the success of one or more non-therapeutic treatments in one or more other users.

Exemplary embodiment 44 is the method according to exemplary embodiment 43, comprising selecting the one or more other users from a plurality of candidates on the basis of a comparison of personal information relating to the user with personal information relating to the candidates.

Exemplary embodiment 45 is the method according to any one of exemplary embodiments 31 to 44, including storing the determined respiratory-air load.

Exemplary embodiment 46 is the method according to any one of exemplary embodiments 31 to 45, including storing the determined respiratory-air load on a server.

In accordance with a further exemplary embodiment a device for evaluating a bad breath measurement is provided and is designed to receive measurement information regarding a VOC concentration in the breath from a measuring device and to compare the measurement information with predefined threshold values and to determine a non-therapeutic treatment on the basis of the comparison.

It should be noted that exemplary embodiments that are applicable in conjunction with the method according to FIG. 3 are applicable analogously for the assembly according to FIG. 4 and the method according to FIG. 5, and vice versa. In addition, the exemplary embodiments can be combined with one another arbitrarily.

The various method steps and the components of the assembly can be realised by one or more circuits. In one embodiment a “circuit” is understood to mean any unit that implements a logic and can be hardware, software, firmware or a combination thereof. Thus, a “circuit” in one embodiment can be a hard-wired logic circuit or a programmable logic circuit, for example a programmable processor, for example a microprocessor. A “circuit” can also be understood to mean a processor which runs software, for example any type of computer program, for example a computer program in programming code for a virtual machine, for example a Java computer program. A “circuit” in one embodiment can be understood to mean any type of implementation of the functions described in further detail.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for determining a non-therapeutic treatment of an oral cavity, the method comprising:
measuring volatile organic constituents in air exhaled from the oral cavity;
determining a respiratory-air load on the basis of the measured concentration; and
determining a non-therapeutic treatment of the oral cavity on the basis of the determined respiratory-air load.

2. The method according to claim 1, wherein the respiratory-air load is an odour load.

3. The method according to claim 1, wherein the measurement of volatile constituents includes a measurement of a concentration of volatile organic constituents in the exhaled air.

4. The method according to claim 1, wherein the measurement of volatile constituents includes a measurement of a concentration of one or more sulphur compounds and/or nitrogen compounds.

5. The method according to claim 1, wherein the measurement of volatile constituents includes a measurement of a concentration of volatile constituents by means of a redox sensor, a photoionisation detection (PID) sensor, or on the basis of gas chromatography (GC), or a combination of two or more thereof.

6. The method according to claim 1, further comprising displaying a specification of the determined non-therapeutic treatment on a display.

7. The method according to claim 1, wherein the non-therapeutic treatment includes use of an oral hygiene product.

8. The method according to claim 1, wherein the determination of the non-therapeutic treatment includes a determination of a chemical composition of an oral hygiene product.

9. The method according to claim 1, wherein the non-therapeutic treatment is determined on the basis of personal information relating to a user for whom the respiratory-air load is determined.

10. The method according to claim 9, wherein the personal information includes one or more items of information regarding age, gender, height, weight, allergies/allergens, eating habits, drinking habits, smoking habits, oral hygiene habits and used oral hygiene products of the user.

11. The method according to claim 1, wherein the non-therapeutic treatment is determined on the basis of a success of one or more non-therapeutic treatments in one or more other users.

12. The method according to claim 11, comprising selecting the one or more other users from a plurality of candidates on the basis of a comparison of personal information relating to the user with personal information relating to the plurality of candidates.

13. The method according to claim 1, comprising storing the determined respiratory-air load on a server.

14. An assembly for determining a non-therapeutic treatment of an oral cavity, comprising:
a measuring device, which is designed to measure volatile organic constituents in air exhaled from the oral cavity; and
an analysis device, which is designed
to determine a respiratory-air load on the basis of the measured concentration, and
to determine a non-therapeutic treatment of the oral cavity on the basis of the determined respiratory-air load.

15. A method for monitoring the success of a non-therapeutic treatment of an oral cavity, comprising:
repeatedly carrying out, during the non-therapeutic treatment of the oral cavity,
measurement of volatile organic constituents in air exhaled from the oral cavity; and
determination of a respiratory-air load on the basis of the measured concentration;
and
determining the success of the non-therapeutic treatment on the basis of the determined respiratory-air loads.
